(19) United States
(12) Patent Application Publication
(10) Pub. No.: US 2026/0265741 A1
(43) Pub. Date: Sep. 10, 2026

(54) MODULATING THE EXPRESSION AND/OR ACTIVITY OF GAS7 FOR MODULATING VIRAL REPLICATION

(71) Applicants: INSTITUT NATIONAL DE LA SANTÉ ET DE LA RECHERCHE MÉDICALE, Paris (FR); INSTITUT CURIE, Paris (FR); UNIVERSITÉ PARIS CITÉ, Paris (FR); CENTRE NATIONAL DE LA RECHERCHE SCIENTIFIQUE, Paris (FR)

(72) Inventors: Philippe BENAROCH, Paris (FR); Vasco RODRIGUES, Paris (FR); Anaël HANOUNA, Paris (FR); Pierre-Grégoire COULON, Paris (FR)

(21) Appl. No.: 19/165,872

(22) PCT Filed: Mar. 22, 2024
(86) PCT No.: PCT/EP2024/057843

§ 371 Date: Sep. 16, 2025

(30) Foreign Application Priority Data
Mar. 23, 2023 (EP) 23305394.1

(51) Int. Cl. C12N 15/113

(52) U.S. Cl. CPC C12N 15/113; C12N 2310/531

(57) ABSTRACT

Macrophages (MØ) are critical for the detection and clearance of pathogens and yet also serve as replication niches for multiple infectious agents. This delicate balance between viral replication and antiviral response remains poorly understood at the molecular level. Addressing this question is of physiological importance given the ever-present threat posed by emerging viral pathogens. Now, the inventors show that the expression of GAS7 in macrophages limits the replication of viral pathogens belonging to all the major viral groups. They show that the antiviral activity of GAS7 is present even in conditions where the classical antiviral response mediated by type I Interferon is neutralized. In particular, the inventor shows that in human monocyte-derived macrophages, silencing GAS7 boosts the replication of multiple viral pathogens representative of the most relevant viral groups. These include the retroviruses HIV-1 and HIV-2, the RNA viruses ZIKA (RNAss+), SINDBIS (RNAss+), Sendai (RNAss−), VSV (RNAss−) and Measles (RNAss−), and the DNA virus HSV-1 (DNA ds). Importantly, the antiviral activity of GAS7 is present even in conditions where type I Interferon is neutralized, by the addition of the B18R protein (which blocks efficiently type I receptor). Moreover, the inventors show that enforcing the over-expression of GAS7 by macrophages further protects them against HIV-1 infection, compared with cells expressing normal levels of this factor. Accordingly, the present invention relates to the modulation of the expression and/or activity of GAS7 for modulating viral replication in a population of macrophages.

### FIELD OF THE INVENTION

The present invention is in the field of medicine, in particular virology.

### BACKGROUND OF THE INVENTION

An antiviral therapy with broad activity against multiple viruses may be lifesaving for patients particularly if no targeted therapies are available, as is the case for certain viral pathogens. While the administration of recombinant type I Interferon may act as a broad antiviral treatment, it is plagued by unwanted side-effects. In this context, activating an alternative cell intrinsic antiviral mechanism may achieve viral control without excessive inflammation.

Macrophages (MØ) are critical for the detection and clearance of pathogens and yet also serve as replication niches for multiple infectious agents. This delicate balance between viral replication and antiviral response remains poorly understood at the molecular level. Addressing this question is of physiological importance, given the ever-present threat posed by emerging viral pathogens. Also, patients afflicted with infections by known human viruses, such as HIV, remain at increased risk of developing pathologies arising from residual viral replication, in the context of antiretroviral therapy.

Viral replication is the process by which a virus (DNA or RNA) hijacks and uses the machinery of the cell it infects to multiply. Type I interferons (IFNs) have been shown to be the most important innate antiviral cytokines of vertebrates. Almost every cell in the body responds to IFN exposure by the rapid induction of a complex transcriptional program involving more than 300 IFN-stimulated genes (ISGs) that makes the cell refractory to virus replication. Most cells can also respond to viral infection by secreting IFNs, warning the neighboring cells, and inhibiting viral spread. However, each vertebrate species is still infected by multiple viruses despite having an intact IFN response. Virus survival depends on its ability to replicate and propagate in the host, which in turn requires viral mechanisms of evasion or subversion of the host IFN response. The ways viruses counteract the host IFN system are diverse and represent critical determinants of virulence.

There is thus still a need for identifying new host targets which would encompass a broad spectrum of action. Especially, there is a need for antiviral agents which would be effective in inhibiting viral replication, independently of the interferon pathway. There is also a need for antiviral agents which would be safe i.e., not toxic with a long-term administration. Such antiviral agents would be effective especially for a long-term treatment, notably effective thanks to the penetration of said antiviral agents into the different lymphoid organs-such as peripheral lymphoid organs and mesenteric ganglions, which are viral reservoirs.

Growth arrest-specific 7 (GAS7) is expressed primarily in terminally differentiated brain cells and predominantly in mature cerebellar Purkinje neurons. GAS7 was initially described as playing a putative role in neuronal development. Diseases associated with GAS7 include Glaucoma, Primary Open Angle and Glaucoma, Normal Tension. Gene Ontology (GO) annotations related to this gene include DNA-binding transcription factor activity and actin filament binding. Gas7 is also expressed in myeloid cells, including dendritic cells and macrophages. However, the role of GAS7 for controlling viral infection has never been investigated.

### SUMMARY OF THE INVENTION

The present invention is defined by the claims. In particular, the present invention relates to the modulation of the expression and/or activity of GAS7 for modulating viral replication in a population of macrophages.

### DETAILED DESCRIPTION OF THE INVENTION

The inventors show that GAS7 allows macrophages to control viral infections even in the absence of a functional type I Interferon response. This suggests that expression of GAS7 confers cells protection against ongoing viral replication.

Accordingly, the first object of the present invention relates to a method of modulating viral replication in a population of cells of a subject comprising the step of modulating the expression and/or activity of GAS7 in said population of cells.

In some embodiments, the subject can be human or any other animal (e.g., birds and mammals) (e.g., domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, the subject is a non-human animal. In some embodiments, the subject is a farm animal or pet. In some embodiments, the subject is a human. In some embodiments, the subject is a human infant. In some embodiments, the subject is a human child. In some embodiments, the subject is a human adult. In some embodiments, the subject is an elderly human. In some embodiments, the subject is a premature human infant.

In some embodiments, the population of cells is a population of macrophages.

In some embodiments, the population of cells is a population of dendritic cells.

In some embodiments, the population of cells is a population of neurons.

As used herein, the term “viral replication” includes the totality of the steps of the replication cycle of the virus. Especially this term includes the main steps of replication of the retroviruses described in the present application, including entry of the virus into the cell, integration of the viral genome into the DNA of the host cell, and viral maturation.

The viruses that fall within the scope of the present invention include DNA viruses and RNA viruses (riboviruses), in particular viruses responsible for cell deficiencies such as immune deficiencies (such as AIDS), respiratory deficiencies (such as SARS and SARS-CoV-2), neuronal deficiencies (such as rabies) or epithelial deficiencies (such as haemorrhagic fevers). More specifically, said virus is a virus selected from the following families:
the coronaviridae, in particular the genus coronavirus, for example, the SARS virus or the SARS-CoV-2 virus;
the retroviruses, in particular those of the genus lentivirus and those of the genus oncovirus, for example the HTLV-1 virus;
the flaviviridae, in particular those of the genus flavivirus, which includes especially the dengue virus, the zika virus, the yellow fever virus and the viruses responsible for viral encephalitis, such as the West Nile virus, the Japanese encephalitis virus and the Saint-Louis encephalitis virus; or in particular those of the genus hepacivirus, such as Hepatitis C virus;
the orthomyxoviruses, which include the influenza viruses (such as H1N1, H1N2, H2N2, H2N3, H3N1, H3N2, H3N8, H5N1, H5N2, H5N3, H5N6, H5N8, H5N9, H6N1, H6N2, H7N1, H7N2, H7N3, H7N4, H7N7, H7N9 and H10N7);
the paramyxoviridae, in particular those of the genus morbillivirus, especially the measles virus, and the respiratory viruses including sendai virus (SeV) and human parainfluenza viruses and, in particular those of the genus pneumovirus, for example human respiratory syncytial virus and metapneumovirus;
the reoviridae, in particular the virus of the genus rotavirus;
the picornaviridae, in particular the viruses of the genus enterovirus, including the polioviruses and the viruses responsible for viral meningitis and the non-enveloped virus Coxsackie virus, those of the genus aphthovirus, especially the aphthous fever virus, and those of the genus rhinovirus; or in particular the viruses of the genus hepatovirus such as Hepatitis A virus;
the filoviridae, in particular the Ebola virus or the Marburg virus;
the arenaviridae, in particular the Lassa virus;
the rhabdoviridae, in particular those of the genus rhabdovirus, including the rabies virus, and the genus vesiculovirus, which includes the vesicular stomatitis virus; the togaviridae, in particular of the genus Rubivirus, including the rubella virus and the genus alphavirus, including the sindbis virus; the poxviridae, in particular the vaccinia and variola viruses;
the herpesviridae, in particular the Herpes type 1 or 2 (HSV-1 or HSV-2), varicella and Zoster viruses; and the hepadnaviridae such as the hepatitis B virus; the hepatitis D virus;
or the hepeviridae such as the Hepatitis E virus.

In some embodiments, the virus is a human retrovirus, in particular a human lentivirus, more particularly a human immunodeficiency (HIV) virus such as HIV-1 or HIV-2, and preferably HIV-1. In some embodiments, the virus is a simian retrovirus, in particular a simian lentivirus, and more particularly a simian immunodeficiency virus (SIV) such as the SIVmac251 or SIVmac239 virus. In some embodiments, the virus is a human herpesvirus, in particular a herpes simplex virus (HSV) such as HSV-1 or HSV-2. In some embodiments, the virus is a measle virus. In some embodiments, the virus is a sindbis virus, zika virus or a vesicular stomatitis virus. In some embodiments, the virus is a orthomyxoviruse, in particular an influenza virus. In some embodiments, the virus is a Coxsackie virus.

A further object to the present invention relates to a method for decreasing the replication capacity of a virus in a population of cells comprising the step of increasing the expression and/or activity of GAS7 in said population of cells.

The expression “decreasing replication capacity” as used herein with reference to a viral phenotype, means that the virus grows to a lower titer when the step of increasing the expression and/or activity of GAS7 is carried out in comparison to the same virus grown when the step of increasing the expression and/or activity of GAS7 is not carried out. In some embodiments, the ability of the virus to replicate in the population of cells is decreased by at least about 10%, or by at least about 20%, or by at least about 30%, or by at least about 40%, or by at least about 50%, or by at least about 60%, or by at least about 70%, or by at least about 80%, or by at least about 90%, or by at least about 100%, or by at least about 200%), or by at least about 300%, or by at least about 400%, or by at least about 500%.

In some embodiments, the ability to prevent, reduce and/or inhibit viral replication is carried-out in vitro. The prevention or inhibition of viral replication can be either partial or total.

In some embodiments, the ability to prevent, reduce and/or inhibit viral replication is carried-out in vivo. The prevention or inhibition of viral replication can be either partial or total. In some embodiments, the method herein disclosed is carried-out to prevent, reduce and/or inhibit viral replication in a subject infected by a virus, or susceptible to be infected by a virus. In particular, the method is particularly suitable for the prophylaxis and/or treatment of viral infections.

As used herein, the expressions “viral infection” and “infected by a virus” mean that said animal or human has been exposed to a pathogenic RNA or DNA virus and that said virus has attached itself to one or more cells of the host and has then penetrated (or is likely to penetrate) into said cell(s) and has had (or will possibly have) harmful effects for at least one cell of said animal or human. In particular, such a viral infection is capable of evolving into clinical signs of induced pathologies or pathologies accompanying said infection. Accordingly, a “viral infection” within the scope of the present invention includes the earliest phases of viral contamination as well as the latest phases and the intermediate phases of viral contamination. By way of example, in the case of HIV, the infection evolves in several phases which may follow one another over time. Four phases in particular are distinguished: (1) the primary infection corresponds to the phase of seroconversion which follows contamination and is (in 50 to 75% of cases) or is not accompanied by symptoms; it is followed by (2) a latent phase, then (3) a phase with minor symptoms, and finally (4) the phase of profound immunodepression or the AIDS stage, which is generally symptomatic and is generally accompanied by numerous opportunistic infections. The term “viral infection” therefore also includes any clinical sign, symptom or disease that occurs in an animal or human (patient) following contamination of said animal or patient by a virus as described in the present application. Accordingly, the “viral infection” includes both contamination by said virus and the various pathologies which are the consequence of contamination by said virus.

The viral infections which fall within the scope of the present invention include in particular the group constituted by viral encephalitis, viral meningitis, aphthous fever, influenza, yellow fever, respiratory viral infections such as infections due to SARS or SARS-CoV-2, which include in particular coronavirus disease-19 (COVID-19), infantile diarrhoea, in particular infantile diarrhoea caused by rotavirus, haemorrhagic fevers, in particular haemorrhagic fevers caused by the Ebola virus, the dengue virus and the Lassa virus, poliomyelitis, rabies, measles, rubella, varicella, smallpox, herpes zoster, genital herpes, hepatitis, especially A, B, C, D and E, leukaemia and paralysis due to HTLV-1 (human T lymphotropic virus type 1), as well as infections caused by an HIV virus, and more particularly by HIV-1 or HIV-2, or an SIV virus, which include in particular acquired immunodeficiency syndrome (AIDS). In particular, the viral infection is a brain viral infection.

As used herein, the term “prophylaxis” or “prevent a viral infection” denotes any degree of retardation in the time of appearance of clinical signs or symptoms of the viral infection, as well as any degree of inhibition of the severity of the clinical signs or symptoms of the viral infection, including, but not being limited to, the total prevention of the viral infection. This requires that the method herein discloses is carried-out in the subject likely to be contaminated by a virus before any clinical sign or symptom of the disease appears. The prophylactic intervention can take place before said animal or human is exposed to the virus responsible for the viral infection, or at the time of exposure. Such a prophylactic administration serves to prevent and/or reduce the severity of any subsequent infection.

As used herein, the term “treatment” is understood as meaning the therapeutic effect produced on an animal or human by the active substances when they are administered to said animal or human at the time of contamination of said animal or human by the virus or after contamination. The method herein disclosed can be carried-out during the primary infection phase, during the asymptomatic phase or after the appearance of clinical signs or symptoms of the disease. In some embodiments, the method herein disclosed is carried-out during the primary infection phase. In some embodiments, the method herein disclosed is carried-out after the primary infection phase, i.e. in the chronic phase (which may be asymptomatic or after the appearance of clinical signs or symptoms of the disease). In some embodiments, the therapeutic intervention takes place within 24 or 48 hours of said animal or human being exposed to said virus, as quickly as possible. Said treatment includes any curative effect obtained by virtue of the implementation of the method herein disclosed, along with the improvement in the clinical signs or symptoms observed in the animal or patient as well as the improvement in the condition of the animal or patient. The term includes, in particular, the effects obtained as a consequence of inhibiting viral replication and/or inhibiting cell death and inflammation induced by the virus. Accordingly, the term “treatment” covers the slowing down, reduction, interruption, and stopping of the viral infection and/or of the harmful consequences of the viral infection; treatment does not necessarily require the complete removal of all the clinical signs of the viral infection and the symptoms of the disease, nor the complete elimination of the virus. The method herein disclosed can be carried-out to an animal or human at risk of developing a viral infection (prophylaxis) or after contamination by the virus has taken place, in particular after manifestation of the first clinical signs or symptoms of the disease, for example after proteins or antibodies specific to the said virus have been detected in the blood of the animal or patient (treatment).

In some embodiments, the method herein disclosed is carried-out in an animal or human before said animal or human is exposed to said virus, during exposure to said virus or after exposure to said virus. Intervention after exposure to the virus can be carried out at any time but will preferably be carried out as quickly as possible after exposure, in particular within 48 hours of the animal or human being exposed to said virus. Furthermore, it is also possible to envisage a plurality of successive interventions for increasing the expression and/or activity of GAS7, so as to increase the beneficial effects of the treatment. In order to increase the chances of cure, or at least prolong the life expectancy of the animal or human, or the prophylactic effect, it is possible in particular to carry out one or more successive interventions before the animal or human is exposed to the virus and/or during exposure to the virus and/or after exposure to the virus, in particular within 48 hours of said animal or human being exposed to said virus.

The method herein disclosed can be used in the prophylaxis and/or treatment of a viral infection, in the primary infection phase and/or in the chronic phase (which may be asymptomatic or after the appearance of clinical signs or symptoms of the disease). The animal or human infected by the virus may be in the primary infection phase or in the chronic phase. The method herein disclosed can also be used to prevent, reduce and/or inhibit viral replication in an animal or human infected by a virus, in the primary infection phase and/or in the chronic phase (which may be asymptomatic or after the appearance of clinical signs or symptoms of the disease).

A further object of the present invention relates to a method for increasing the replication capacity of a virus in a population of cells comprising the step of decreasing the expression and/or activity of GAS7 in said population of cells.

The expression “increasing replication capacity,” as used herein with reference to a viral phenotype, means that the virus grows to a lower titer when the step of increasing the expression and/or activity of GAS7 is carried out in comparison to the same virus grown when the step of increasing the expression and/or activity of GAS7 is not carried out. In some embodiments, the method herein disclosed will increase the ability of the virus to replicate in a cell by at least about 10%, or by at least about 20%, or by at least about 30%, or by at least about 40%, or by at least about 50%, or by at least about 60%, or by at least about 70%, or by at least about 80%, or by at least about 90%, or by at least about 100%, or by at least about 200%), or by at least about 300%, or by at least about 400%, or by at least about 500%.

In some embodiments, the method herein disclosed, can be used for the production of an amount of viruses. In particular, the method herein disclosed is particularly suitable for the production of a vaccine.

In some embodiments, the method herein disclosed comprises the steps consisting of i) infecting said population of cells with said virus and ii) decreasing the expression and/or activity of GAS7 in said population of infected cells.

According to the present invention, any virus strain can be used. Preferably, said virus strain corresponds to a clinical isolate of at least one circulating pathogenic strain of a virus. In some embodiments, the clinical isolate can be made into a high-growth strain by reassortment with a high-growth master donor strain, or by multiple passages of the clinical isolate in continuous mammalian cell lines, with a selection of high-growth variants.

Typically, the infection of the cells with influenza viruses is carried out at an m.o.i. (multiplicity of infection) of about 0.0001 to 10, preferably of 0.002 to 0.5.

According to the invention, any population of macrophages, dendritic cells or neurons may be used. For instance, mammalian populations of cells include but are not limited to cells from humans, dogs, cats, cattle, horses, sheep, pigs, goats, and rabbits. In some embodiments, the population of cells is a human population of cells. In some embodiments, the cell is a cell line. Typically, the cell is certified according to the WHO requirements for vaccine production. The requirements for certifying such cell lines include characterization with respect to at least one of genealogy, growth characteristics, immunological markers, virus susceptibility tumorigenicity and storage conditions, as well as by testing in animals, eggs, and cell culture. It is preferred to establish a complete characterization of the cell line to be used. Data that can be used for the characterization of a cell line to be used in the present invention includes (a) information on its origin, derivation, and passage history; (b) information on its growth and morphological characteristics; (c) distinguishing features, such as biochemical, immunological, and cytogenetic patterns which allow the cells to be clearly recognized among other cell lines; and (d) results of tests for tumorigenicity. Preferably, the passage level, or population doubling, of the cell line used is as low as possible. Examples of macrophage cells lines that can be suitable of implementing the present invention include the IPKM cell line (Masujin, K., Kitamura, T., Kameyama, K. i. et al. An immortalized porcine macrophage cell line competent for the isolation of African swine fever virus. Sci Rep 11, 4759 (2021)) or the RAW 264.7 cell line (Taciak B, Białasek M, Braniewska A, Sas Z, Sawicka P, Kiraga Ł, Rygiel T, Król M. Evaluation of phenotypic and functional stability of RAW 264.7 cell line through serial passages. PLoS One. 2018 Jun. 11; 13 (6):e0198943).

Typically, the population of cells is cultured in a standard commercial culture medium, such as Dulbecco's modified Eagle's medium supplemented with serum (e.g., 10% foetal bovine serum), or in serum free medium, under controlled humidity and C0₂ concentration suitable for maintaining neutral buffered pH (e.g., at pH between 7.0 and 7.2). Optionally, the medium contains antibiotics to prevent bacterial growth, e.g., penicillin, streptomycin, etc., and/or additional nutrients, such as L-glutamine, sodium pyruvate, nonessential amino acids, additional supplements to promote favourable growth characteristics.

The population of cells for production of the virus can be cultured in serum-containing or serum free medium. In some case, e.g., for the preparation of purified viruses, it is desirable to grow the cells in serum free conditions. Cells can be cultured in small scale, e.g., less than 25 ml medium, culture tubes or flasks or in large flasks with agitation, in rotator bottles, oronmicrocarrierbeads (e.g., DEAE-Dextran microcarrier beads, such as Dormacell, Pfeifer & Langen; Superbead, Flow Laboratories; styrene copolymer-tri-methylamine beads, such as Hillex, SoloHill, Ann Arbor) in flasks, bottles or reactor cultures. Microcarrier beads are small spheres (in the range of 100-200 microns in diameter) that provide a large surface area for adherent cell growth per volume of cell culture. For example, a single liter of medium can include more than 20 million microcarrier beads providing greater than 8000 square centimeters of growth surface. For commercial production of viruses, e.g., for vaccine production, it is often desirable to culture the cells in a bioreactor or fermenter. Bioreactors are available in volumes from under 1 liter to in excess of 100 liters, e.g., Cyto3 Bioreactor (Osmonics, Minnetonka, Minn.); NBS bioreactors (New Brunswick Scientific, Edison, N.J.); laboratory and commercial scale bioreactors from B. Braun Biotech International (B. Braun Biotech, Melsungen, Germany).

The cells can be grown in culture under conditions permissive for replication and assembly of viruses. In some embodiments, cells can be cultured at a temperature below about 37° C., preferably at a temperature equal to, or less than, about 35° C. The culturing of the cells is carried out as a rule at a regulated pH which is preferably in the range from pH 6.6 to pH 7.8, in particular in the range from pH 6.8 to pH 7.3. Furthermore, the pO2 value can advantageously be regulated and is then as a rule between 25% and 95%, in particular between 35% and 60% (based on the air saturation).

Following culture for a suitable period of time to permit replication of the virus to high titer, the virus can be recovered. Viruses can typically be recovered from the culture medium, in which infected (transfected) cells have been grown. Typically, crude medium is clarified prior to concentration of viruses. Common methods include filtration, ultrafiltration, gradient ultracentrifugation, adsorption on barium sulfate and elution, and centrifugation. For example, crude medium from infected cultures can first be clarified by centrifugation at, e.g., 1000-2000×g for a time sufficient to remove cell debris and other large particulate matter, e.g., between 10 and 30 minutes. Alternatively, the medium is filtered through a 0.8 μm cellulose acetate filter to remove intact cells and other large particulate matter. Optionally, the clarified medium supernatant is then centrifuged to pellet the viruses, e.g., at 15,000×g, for approximately 3-5 hours. Following resuspension of the virus pellet in an appropriate buffer, such as STE (0.01 MTris-HCl; 0.15 MNaCl; 0.0001 MEDTA) or phosphate buffered saline (PBS) at pH 7.4, the virus is concentrated by density gradient centrifugation on sucrose (60%-12%) or potassium tartrate (50%-10%). Either continuous or step gradients are suitable, e.g., a sucrose gradient between 12% and 60% (in four 12% steps). The gradients are centrifuged at a speed, and for a time, sufficient for the viruses to concentrate into a visible band for recovery. Alternatively, and for most large-scale commercial applications, the virus is elutriated from density gradients using a zonal-centrifuge rotor operating in continuous mode. If desired, the recovered viruses can be stored at −80° C. in the presence of sucrose-phosphate-glutamate (SPG) as a stabilizer.

The method of the present invention is particularly useful for the production of virus vaccines. The resulting replicated virus can be indeed concentrated as above described and then be inactivated or attenuated using any method well known in the art. Inactivated virus vaccines of the invention are typically provided by inactivating replicated virus of the invention using known methods, such as, but not limited to, formalin or beta-propiolactone treatment. Inactivated vaccine types that can be used in the invention can include whole-virus (WV) vaccine or subvirion (SV) virus vaccine. The WV vaccine contains intact, inactivated virus, while the SV vaccine contains purified virus disrupted with detergents that solubilize the lipid-containing viral envelope, followed by chemical inactivation of residual virus. Live, attenuated virus vaccines, using replicated virus of the invention, can also be used for preventing or treating viral infection, according to known method steps: attenuation is preferably achieved in a single step by transfer of attenuating genes from an attenuated donor virus to a replicated isolate or reassorted virus according to known methods. Other attenuating mutations can be introduced into virus genes by site-directed mutagenesis to rescue infectious viruses bearing these mutant genes. Attenuating mutations can be introduced into non-coding regions of the genome, as-well as into coding regions. Thus, new donor viruses can also be generated bearing attenuating mutations introduced by site-directed mutagenesis. It is preferred that such attenuated viruses maintain the genes from the replicated virus that encode antigenic determinants substantially similar to those of the original clinical isolates. This is because the purpose of the attenuated vaccine is to provide substantially the same antigenicity as the original clinical isolate of the virus, while at the same time lacking infectivity to the degree that the vaccine causes minimal chance of inducing a serious pathogenic condition in the vaccinated mammal.

The replicated virus that is attenuated or inactivated may be then formulated in a vaccine composition. Vaccine compositions of the present invention, suitable for inoculation or for parenteral or oral administration, comprise attenuated or inactivated viruses, optionally further comprising sterile aqueous or non-aqueous solutions, suspensions, and emulsions. The composition can further comprise auxiliary agents or excipients, as known in the art. When a vaccine composition of the present invention is used for administration to an individual, it can further comprise salts, buffers, adjuvants, or other substances which are desirable for improving the efficacy of the composition. Adjuvants are substances that can be used to augment a specific immune response. Normally, the adjuvant and the composition are mixed prior to presentation to the immune system, or presented separately, but into the same site of the mammal being immunized.

As used herein, the term “GAS7” has its general meaning in the art and refers to the Growth arrest-specific protein 7 encoding by GAS7 gene. The term is also known as KIAA0394 or MGC1348. An exemplary amino acid sequence for GAS7 is represented by SEQ ID NO:1.

>sp|O60861|GAS7 HUMAN Growth arrest-specific
protein 7
OS = Homo sapiens
OX = 9606 GN-GAS7 PE = 1 SV = 3
SEQ ID NO: 1
MSGARCRTLYPFSGERHGQGLRFAAGELITLLQVPDGGWWEGEKED
GLRGWFPASYVQLLEKPGMVPPPPGEESQTVILPPGWQSYLSPQG
RRYYVNTTTNETTWERPSSSPGIPASPGSHRSSLPPTVNGYHASG
TPAHPPETAHMSVRKSTGDSQNLGSSSPSKKQSKENTITINCVTF
PHPDTMPEQQLLKPTEWSYCDYFWADKKDPQGNGTVAGFELLLQK
QLKGKQMQKEMSEFIRERIKIEEDYAKNLAKLSQNSLASQEEGSL
GEAWAQVKKSLADEAEVHLKESAKLHSEVEKPLMNFRENFKKDMK
KCDHHIADLRKQLASRYASVEKARKALTERQRDLEMKTQQLEIKL
SNKTEEDIKKARRKSTQAGDDLMRCVDLYNQAQSKWFEEMVTTTL
ELERLEVERVEMIRQHLCQYTQLRHETDMFNQSTVEPVDQLLRKV
DPAKDRELWVREHKTGNIRPVDMEI

Typically, the modulation of the expression and/or activity of GAS7 can be performed by any means well-known in the art.

In some embodiments, the methods herein disclosed herein implements the use of one or more agent(s) that increase the expression and/or activity of GAS7.

In some embodiments, the methods herein disclosed herein implements the use of one or more agent(s) that decrease the expression and/or activity of GAS7.

As used herein, the term “agent” means chemical compounds, mixtures of chemical compounds, biological macromolecules, or extracts made from biological materials, such as bacteria, plants, fungi, or animal particularly mammalian) cells or tissues that are suspected of having therapeutic properties. According to the present invention the term “agent” includes GAS7 itself, a variant thereof (e.g. a negative dominant) or a polynucleotide that encodes for GAS7 or for a variant thereof. The agent may be purified, substantially purified, or partially purified. The expression and/or activity of GAS7 can be measured by any assay well known in the art and typically include those described in the EXAMPLE.

As used herein, the expression “agent that increases the expression and/or activity of GAS7” refers to any agent that is capable of increasing the expression and/or activity of GAS7 in the population of cells by at least about 10%, or by at least about 20%, or by at least about 30%, or by at least about 40%, or by at least about 50%, or by at least about 60%, or by at least about 70%, or by at least about 80%, or by at least about 90%, or by at least about 100%, or by at least about 200%, or by at least about 300%, or by at least about 400%, or by at least about 500% when compared to the expression and/or activity that is measured in the absence of the agent. On the contrary, the expression “agent that decreases the expression and/or activity of GAS7” refers to any agent that is capable of decreasing the expression and/or activity of GAS7 in the population of cells by at least about 10%, or by at least about 20%, or by at least about 30%, or by at least about 40%, or by at least about 50%, or by at least about 60%, or by at least about 70%, or by at least about 80%, or by at least about 90%, or by at least about 100%, or by at least about 200%, or by at least about 300%, or by at least about 400%, or by at least about 500% when compared to the expression and/or activity that is measured in the absence of the agent.

In some embodiments, the agent is a small organic molecule. The term “small organic molecule” as used herein, refers to any molecule of a size comparable to those organic molecules generally used in pharmaceuticals. The term excludes biological macromolecules (e.g., proteins, nucleic acids, etc.). Preferred small organic molecules range in size from approximately 10 Da up to about 5000 Da, more preferably up to 2000 Da, and most preferably up to about 1000 Da.

In some embodiments, the agent that increases the expression and/or activity of GAS7 is either i) a polypeptide comprising an amino acid sequence having at least 90% of identity with the amino acid sequence as set forth in SEQ ID NO:1 or ii) a polynucleotide encoding for such a polypeptide.

In some embodiments, the agent that decreases the expression and/or activity of GAS7 is either i) a polypeptide comprising an amino acid sequence having at least 90% of identity with the amino acid sequence as set forth in SEQ ID NO:1 or ii) a polynucleotide encoding for such a polypeptide is provided that said polypeptide or polynucleotide is a dominant negative polypeptide or polynucleotide of GAS7.

As used herein, the term “polypeptide” has its general meaning in the art and refers to a polymer of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention, a first amino acid sequence having at least 90% of identity with a second amino acid sequence means that the first sequence has 90; 91; 92; 93; 94; 95; 96; 97; 98; 99 or 100% of identity with the second amino acid sequence.

In some embodiments, the polypeptide (including the dominant negative polypeptide) is fused to a heterologous moiety. In some embodiments, the heterologous moiety is a cell-penetrating peptide. As used herein, the term “cell-penetrating peptide” refers to a short peptide, for example comprising from 5 to 50 amino acids, which can readily cross biological membranes and is capable of facilitating the cellular uptake of various molecular cargos, in vitro and/or in vivo. In some embodiments, the heterologous polypeptide is an internalization sequence derived either from the homeodomain of Drosophila Antennapedia/Penetratin (Antp) protein (amino acids 43-58) or the trans-activating transcriptional activator of HIV-1.

As used herein, the term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some embodiments, the polynucleotide comprises an mRNA. In other aspect, the mRNA is a synthetic mRNA. In some embodiments, the synthetic mRNA comprises at least one unnatural nucleobase. In some embodiments, all nucleobases of a certain class have been replaced with unnatural nucleobases (e.g., all uridines in a polynucleotide disclosed herein can be replaced with an unnatural nucleobase, e.g., 5-methoxyuridine). In some embodiments, the polynucleotide (e.g., a synthetic RNA or a synthetic DNA) comprises only natural nucleobases, i.e., A, C, T and G in the case of a synthetic DNA, or A, C, T, and U in the case of a synthetic RNA.

As used herein, the term “dominant negative” in the context of protein mechanism of action or gene phenotype, refers to a mutant or variant polypeptide, or the polynucleotide encoding the mutant or variant polypeptide, that substantially prevents a corresponding polypeptide having wild-type function from performing the wild-type function.

In some embodiments, the polynucleotide of the present invention is a messenger RNA (mRNA).

In some embodiments, the polynucleotide is inserted in a vector, such as a plasmid, cosmid, episome, artificial chromosome, phage or a viral vector. Typically, the vector is a viral vector which is an adeno-associated virus (AAV), a retrovirus, bovine papilloma virus, an adenovirus vector, a lentiviral vector, a vaccinia virus, a polyoma virus, or an infective virus. Typically, the vector of the present invention includes “control sequences”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

In some embodiments, the polypeptide or polynucleotide of the present invention can be conjugated to at least one other molecule. Typically, said molecule is selected from the group consisting of polynucleotides, polypeptides, lipids, lectins, carbohydrates, vitamins, cofactors, and drugs.

In some embodiments, the polypeptide or polynucleotide of the present invention is formulated with lipidoids. The synthesis of lipidoids has been extensively described (see Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001). While these lipidoids have been used to effectively deliver double stranded small interfering RNA molecules in rodents and non-human primates (see Akinc et al., Nat Biotechnol. 2008 26:561-569; Frank-Kamenetsky et al., Proc Natl Acad Sci USA. 2008 105:11915-11920; Akinc et al., Mol Ther. 2009 17:872-879; Love et al., Proc Natl Acad Sci USA. 2010 107:1864-1869; Leuschner et al., Nat Biotechnol. 2011 29:1005-1010), the present disclosure describes their formulation and use in delivering polynucleotides.

In some embodiments, the polypeptide or polynucleotide of the present invention is formulated using one or more lipid-based structures that include but are not limited to liposomes, lipoplexes, or lipid nanoparticles (Paunovska, Kalina, David Loughrey, and James E. Dahlman. “Drug delivery systems for RNA therapeutics.” Nature Reviews Genetics (2022): 1-16).

Liposomes are artificially prepared vesicles which can primarily be composed of a lipid bilayer and can be used as a delivery vehicle for the administration of pharmaceutical formulations. Liposomes can be of different sizes such as, but not limited to, a multilamellar vesicle (MLV) which can be hundreds of nanometers in diameter and can contain a series of concentric bilayers separated by narrow aqueous compartments, a small unicellular vesicle (SUV) which can be smaller than 50 nm in diameter, and a large unilamellar vesicle (LUV) which can be between 50 and 500 nm in diameter. Liposome design can include, but is not limited to, opsonins or ligands in order to improve the attachment of liposomes to unhealthy tissue or to activate events such as, but not limited to, endocytosis. Liposomes can contain a low or a high pH in order to improve the delivery of the pharmaceutical formulations. As a non-limiting example, liposomes such as synthetic membrane vesicles are prepared by the methods, apparatus and devices described in US Patent Publication No. US20130177638, US20130177637, US20130177636, US20130177635, US20130177634, US20130177633, US20130183375, US20130183373 and US20130183372. In some embodiments, the liposomes are formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (as described in US20100324120) and liposomes which can deliver small molecule drugs such as, but not limited to, DOXIL® from Janssen Biotech, Inc. (Horsham, Pa.). The polypeptide of polynucleotide of the present invention can be encapsulated by the liposome and/or it can be contained in an aqueous core which can then be encapsulated by the liposome (see International Pub. Nos. WO2012031046, WO2012031043, WO2012030901 and WO2012006378 and US Patent Publication No. US20130189351, US20130195969 and US20130202684).

In some embodiments, the polynucleotide of the present invention is formulated with stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104). The original manufacture method by Wheeler et al. was a detergent dialysis method, which was later improved by Jeffs et al. and is referred to as the spontaneous vesicle formation method. The liposome formulations are composed of 3 to 4 lipid components in addition to the polynucleotide. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some embodiments, the polynucleotide of the present invention is formulated in a lipid nanoparticle such as those described in International Publication No. WO2012170930. Lipid nanoparticle formulations typically comprise a lipid such as, in particular, an ionizable cationic lipid, and further comprise a neutral lipid, a sterol and a molecule capable of reducing particle aggregation, for example a PEG or PEG-modified lipid. The lipid can be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some embodiments, the lipid is a cationic lipid such as, but not limited to, DLin-DMA, DLin-D-DMA, DLin-MC3-DMA, DLin-KC2-DMA, DODMA and amino alcohol lipids. The amino alcohol cationic lipid can be the lipids described in and/or made by the methods described in US Patent Publication No. US20130150625. As a non-limiting example, the cationic lipid can be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof. Nanoparticle formulations of the present disclosure can be coated with a surfactant or polymer in order to improve the delivery of the particle. In some embodiments, the nanoparticle is coated with a hydrophilic coating such as, but not limited to, PEG coatings and/or coatings that have a neutral surface charge. The hydrophilic coatings can help to deliver nanoparticles with larger payloads such as, but not limited to, polynucleotides within the central nervous system. As a non-limiting example nanoparticles comprising a hydrophilic coating and methods of making such nanoparticles are described in US Patent Publication No. US20130183244.

In some embodiments, the agent that decreases the expression and/or activity of GAS7 is a molecule that partially or fully blocks, inhibits, or neutralizes a biological activity or expression of GAS7. The agent can be a molecule of any type that interferes with the signalling associated with GAS7 in the population of cells, for example, either by decreasing transcription or translation of GAS7-encoding nucleic acid, or by inhibiting or blocking GAS7 polypeptide activity, or both. Examples of agents that decreases the expression and/or activity of GAS7 include, but are not limited to, antisense polynucleotides, interfering RNAs, catalytic RNAs, RNA-DNA chimeras, GAS7-specific aptamers, anti-GAS7 antibodies, GAS7-binding fragments of anti-GAS7 antibodies, GAS7-binding small molecules, GAS7-binding peptides, and other polypeptides that specifically bind GAS7 (including, but not limited to, GAS7-binding fragments of one or more GAS7 ligands, optionally fused to one or more additional domains). Other examples includes the use of endonucleases such as CRISPR-endonucleases (e.g. CAS9) with designed guide RNA molecules for decreasing the expression of GAS7.

A further object of the present invention relates to a method of screening an agent that is capable of decreasing the replication capacity of a virus in a population of cells comprising the steps of i) contacting the population of cells with a plurality of test substances and ii) selecting the substance(s) that is (are) capable of increasing the expression and/or activity of GAS7 in said population of cells.

A further object of the present invention relates to a method of screening an agent that is capable of increasing the replication capacity of a virus in a population of cells comprising the steps of i) contacting the population of cells with a plurality of test substances and ii) selecting the substance(s) that is (are) capable of decreasing the expression and/or activity of GAS7 in said population of cells.

The invention will be further illustrated by the following figures and examples. However, these examples and figures should not be interpreted in any way as limiting the scope of the present invention.

### FIGURES

FIG. 1. Gas7 restrict viral replication of various viruses in macrophages.

A—Schematic representation of the experimental procedure. Monocytes were isolated from human blood and transduced with lentiviral particles encoding for a Control or GAS7-targeted shRNAs. Monocytes were allowed to differentiate into macrophages for 6 days in culture and infected with the indicated viruses. Following infection, kinetics of viral propagations were measured by automated microscopy (Incucyte) and flow cytometry. Viruses were also titered in supernatant (SN) by Vero plaque assay. The decoy IFNAR receptor B18R was added in most conditions as indicated to block type I IFN signalling.

B—left panel (up and down)—HSV-1 (VP26GFP+) propagation in infected macrophages (MOI 1) was assessed by Incucyte, measuring total GFP intensity overtime. Lower panel shows representative Incucyte pictures of the GFP+ infected cells at day 1 post infection. Right panel (up)—Percentage of GFP+ infected cells at 24 hrs post infection (p.i.) measured by flow-cytometry. Right panel (down)—viral titration of supernatant from infected cells (pfu/ml) determined by VERO plaque assay (representative picture of the plaques displayed). All data are representative of independent experiments performed using macrophages differentiated from monocytes sorted from n=5 healthy donors). B18R was added to all the cultures to block type I IFN signalling.

C—left panel (up and down)—Measle (MS GFP+) propagation in infected macrophages (MOI 1) was assessed by Incucyte, measuring total GFP intensity overtime. Lower panel shows representative Incucyte pictures of the GFP+ infected cells at day 3 post infection. Right panel (up)—Percentage of GFP+ infected cells at 48 hrs post infection (p.i.) measured by flow-cytometry. Right panel (down)—viral titration of supernatant from infected cells (pfu/ml) determined by VERO plaque assay (representative picture of the plaques displayed). All data are representative of independent experiments performed using macrophages differentiated from monocytes sorted from n=5 healthy donors). B18R was added to all the cultures to block type I IFN signalling.

D—left panel (up and down)—Sindbis (SINV GFP+) propagation in infected macrophages (MOI 10) was assessed by Incucyte, measuring total GFP intensity overtime. Lower panel shows representative Incucyte pictures of the GFP+ infected cells at day 1 post infection. Right panel (up)—Percentage of GFP+ infected cells at 24 hrs post infection (p.i.) measured by flow-cytometry. Right panel (down)—viral titration of supernatant from infected cells (pfu/ml) determined by VERO plaque assay (representative picture of the plaques displayed). All data are representative of independent experiments performed using macrophages differentiated from monocytes sorted from n=5 healthy donors). B18R was added to all the cultures to block type I IFN signalling.

E-upper panel (right and left)—Vesicular Stomatitis Virus (VSV GFP+) propagation in infected macrophages (MOI 1) was assessed by Incucyte, measuring total GFP intensity overtime. Left panel shows representative Incucyte pictures of the GFP+ infected cells at day 2 post infection. Lower panel—viral titration of supernatants from infected cells (pfu/ml) determined by VERO plaque assay (representative picture of the plaques displayed). All data are representative of independent experiments performed using macrophages differentiated from monocytes sorted from n=5 healthy donors). B18R was added to all the cultures to block type I IFN signalling.

F—HIV-1 (NL4.3 ΔNef GFP+) and HIV-2 (ROD9 ΔNef GFP+) propagation in infected macrophages (MOI 0.2) was assessed by Incucyte, measuring total GFP intensity overtime (graphs on top). Lower panels show representative Incucyte pictures of the GFP+ infected cells at 6 days post infection. Of note, the small difference between GAS7 ShRNA and CTRL ShRNA with regards to HIV-1 propagation is abrogated by the addition of B18R, indicating that in the absence of GAS7 type I IFN is produced in the culture in response to HIV-1. In contrast, addition of B18R to the cultures exposed to HIV-2 has no impact on the kinetics of viral propagation.

G—upper panel—Sendai (WT) propagation in infected macrophages (0.04 HA units/mL) was assessed by microscopy observing syncytia formation and cell mortality (63 hrs p.i.). Lower panel shows qRT-PCR analysis of 3 viral genes expression (Matrix, Large and Phospho-Protein). B18R was added to all the cultures to block type I IFN signalling.

H—upper panel—kinetic of ZIKA (WT) infection (MOI 3) in macrophages was assessed by flow cytometry after intracellular immunostaining for the E protein at days 2,4 and 6 post infection. Lower left panel shows cell mortality and lower right panel viral titration of supernatant from infected cells (pfu/ml) determined by VERO plaque assay (representative picture of the plaques displayed). All data are representative of independent experiments performed using macrophages differentiated from monocytes sorted from n=5 healthy donors). B18R was added to all the cultures to block type I IFN signalling. B18R was added to all the cultures to block type I IFN signalling.

I—upper panel—kinetic of Influenza (H3N2) infection (MOI 0.5) in macrophages was assessed by flow cytometry after intracellular immunostaining for the HA protein at 8, 16, and 24 hrs post infection. Lower left panel shows % of cell mortality (aqua staining) and lower right panel viral titration of supernatant from infected cells (pfu/ml) determined by VERO plaque assay (representative picture of the plaques displayed). All data are representative of independent experiments performed using macrophages differentiated from monocytes sorted from n=5 healthy donors). B18R was added to all the cultures to block type I IFN signalling.

### EXAMPLE

### Methods

### Cells

Cytapheresis blood residues from healthy adult donors were obtained from the Etablissement Français du Sang (EFS). All donors signed informed consent forms for their blood to be used for research purposes. Following PBMCs separation by Ficoll-Paque gradient centrifugation (GE Healthcare), purified monocyte populations were isolated with CD14⁺ magnetic beads (Miltenyi Biotec). To obtain Monocytes Derived Macrophages (MDMs), cells were differentiated in non-treated Petri dishes for 6 days in MDM media containing 5% FBS complemented RPMI 1640 (Gibco, Life Technologies) plus 5% human serum AB (Sigma), antibiotics, non-essential amino acids, sodium pyruvate (Gibco) and 50 ng/ml Macrophage Colony Stimulating Factor (M-CSF, Miltenyi Biotech).

### Viral Productions

Lentiviral shRNA coding particles were produced by transfection of 293 FT cells in T300 flasks with a plasmid mix consisting of 5 μg CMV-VSVG (pMD2.G; 12259; Addgene), 11 μg of packaging plasmid PSPAX2 (12260; Addgene), 0.6 μg of Vpx-Vpr plasmid (cloned in the lab) and 16 μg of shRNA encoding plasmid (pLKO.1) targeting Gas7 (GCCCAGTCCAAATGGTTTGAA) or scramble control, purchased from Sigma. All plasmids were mixed in 5 ml Opti-MEM media (Gibco) plus 464 μl PEImax transfection reagent (Polysciences) before being added on 293 FT cells for OVN. Transfection media was removed, and MDM media was added 24-48 hours before collection for lentivirus titration using GHOST X4R5 reporter cell line. Lentiviruses were filtered through a 0.45 μm pore and stored at −80° C. until use.

HIV-1 NL4-3 GFP and HIV-2 ROD9 GFP were produced and tittered following a similar protocol, with a plasmid mix of 28.4 μg containing the HIV proviral DNA. HSV-1 VP26-GFP (KOS strain) virus was propagated and amplified in VERO cells. Measles virus (MVSchw-ATU2-GFP strain) was provided by Frederic Tangy laboratory. ZIKA virus (pMR766 molecular clone), Sindbis GFP virus (pTR339 molecular clone) were produced by reverse genetic systems and kindly given by Enzo Poirier. H3N2 Influenza A virus (X31 strain) was given by Sebastian Amigorena's team.

### In Vitro Transductions and Infections of Human Macrophages Derived Monocytes

Monocytes were transduced with 8 ml of harvested lentiviral productions plus 8 μg/mL of protamine (Sigma). After 48 hours, 2 g/mL of puromycin was added to the differentiating macrophage to select successfully transduced cells. Cells were harvested for experiments on day 6 of differentiation.

Transduced macrophages were detached from Petri dishes using accutase (Stemcell Technologies), plated in non-treated 48-wells plates, and infected one day later with either HSV-1 VP26-GFP, Measles GFP or VSV GFP at an MOI of 1. H3N2 Influenza A infection was done at an MOI of 0.5, ZIKA infection at an MOI of 3, and Sindbis GFP infection at an MOI of 10. Infection with all these viruses was carried on following the same protocol: 2 hours incubation in FBS-free RPMI media at 37° C. with intermittent rocking. Cells were then rinsed with PBS 1× and overlaid with fresh MDM media.

Plated MDMs were infected with HIV in MDM media by adding HIV-1 NL4-3-GFP at an MOI of 1.0 or HIV-2 ROD9-GFP at an MOI of 1.0. After 16 hours, culture media was renewed, and the infection was allowed to progress for additional 8 days.

Infection with GFP+ viruses was monitored by measuring the integrated intensity of green fluorescence over time in photos taken every 2 hours by IncuCyte® imaging (Sartorius) at 20× magnification. WT viruses' infection kinetics were performed by flow-cytometry staining at various time points ranging from 6 hours to 6 days p.i. Briefly, infected macrophages were detached following accutase treatment and stained for 1 hour in PBS 1×0.5% BSA, 0.1% sodium azide at 4° C. with the following antibodies: FITC rabbit polyclonal anti-flavivirus and APC-conjugated mouse monoclonal anti-HA. After washing and subsequent fixation in PFA 4%, cell acquisition was performed on the Novocyte 3000 (Agilent). Flow data were analyzed using FlowJo.

### Results:

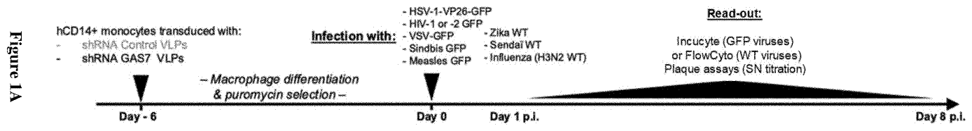

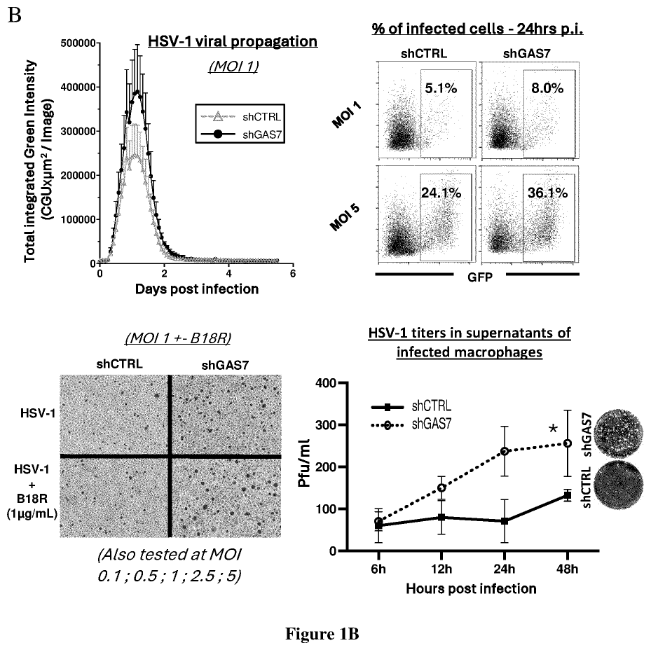

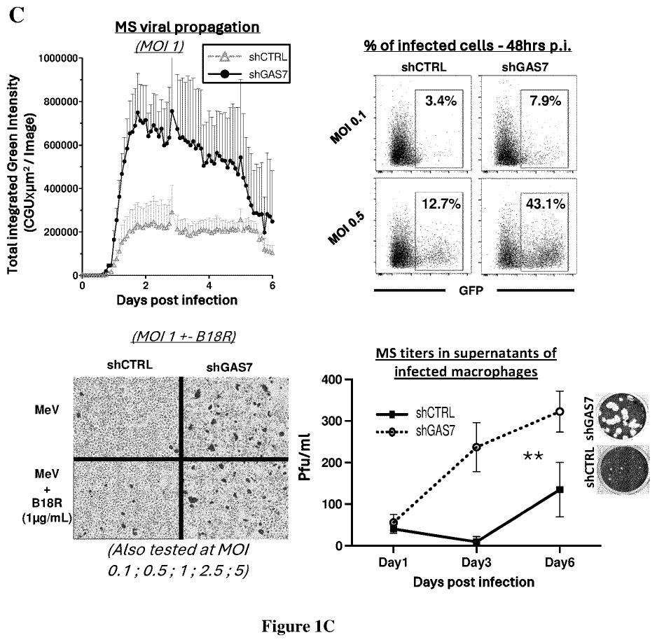

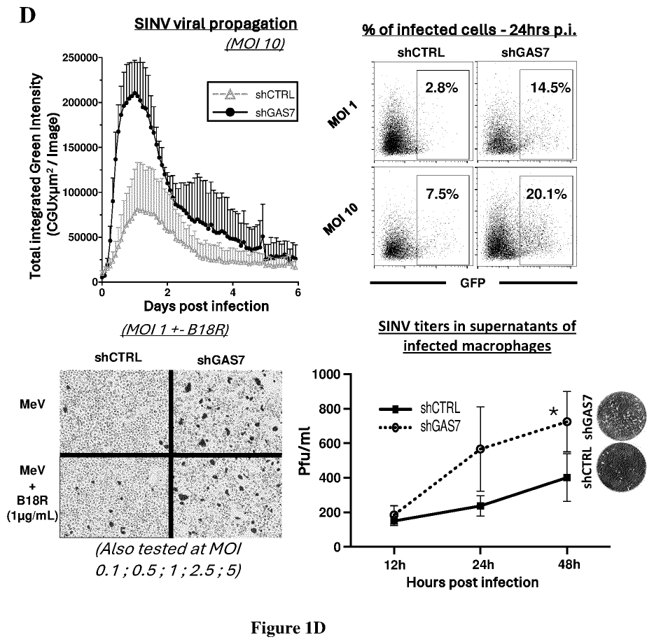

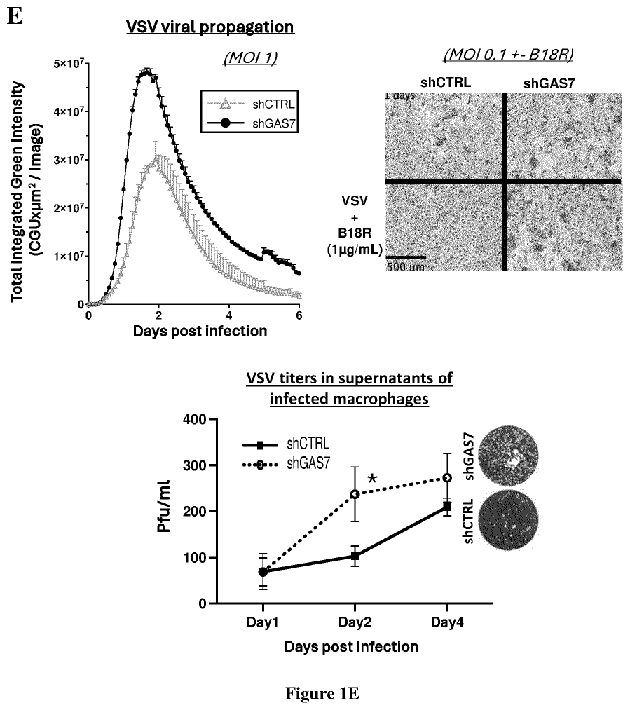

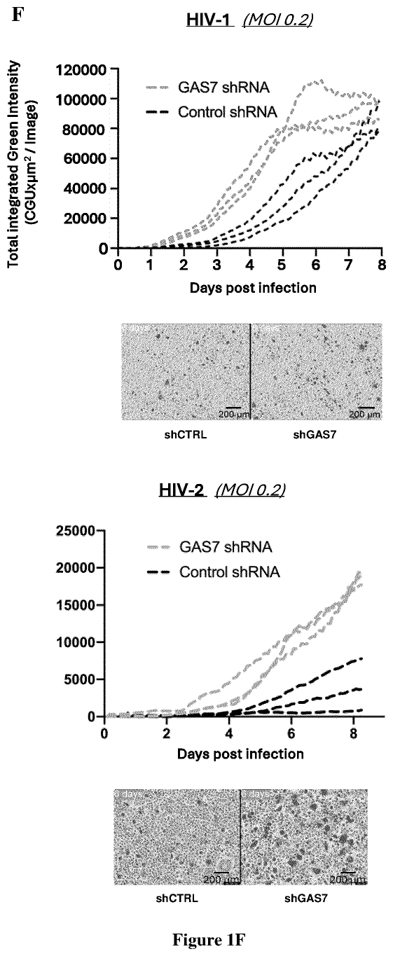

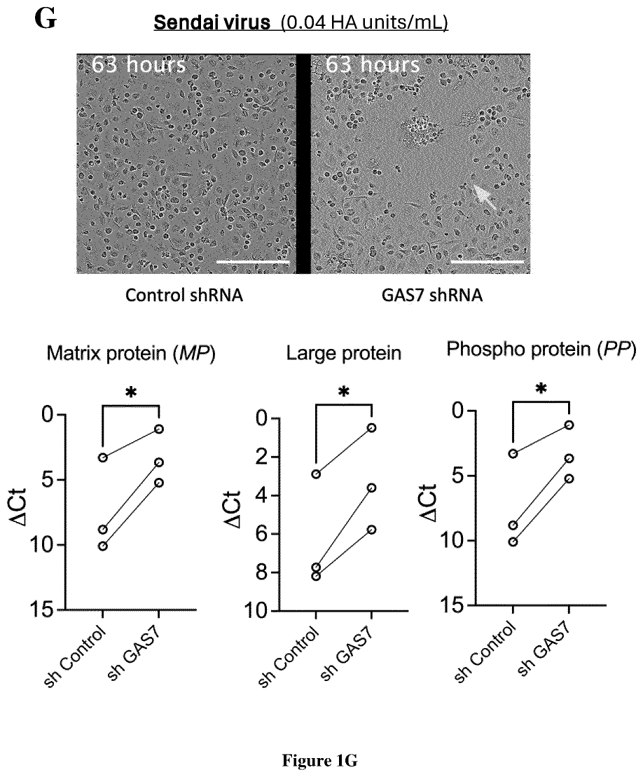

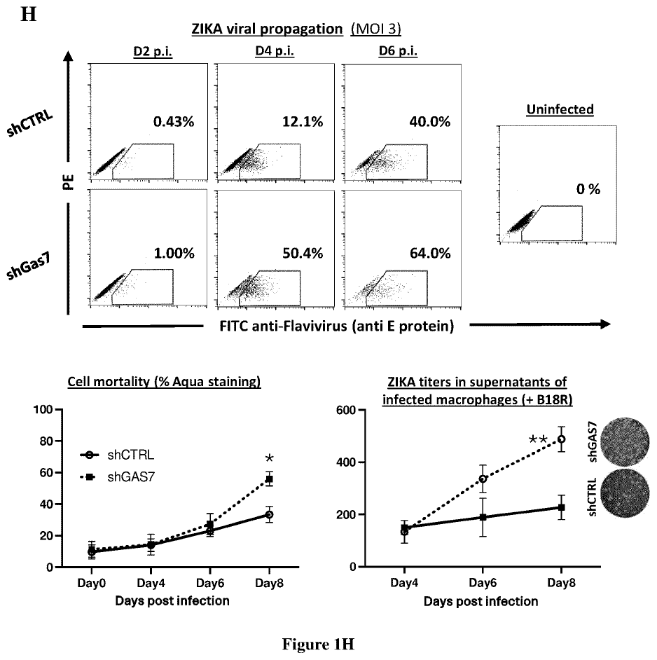

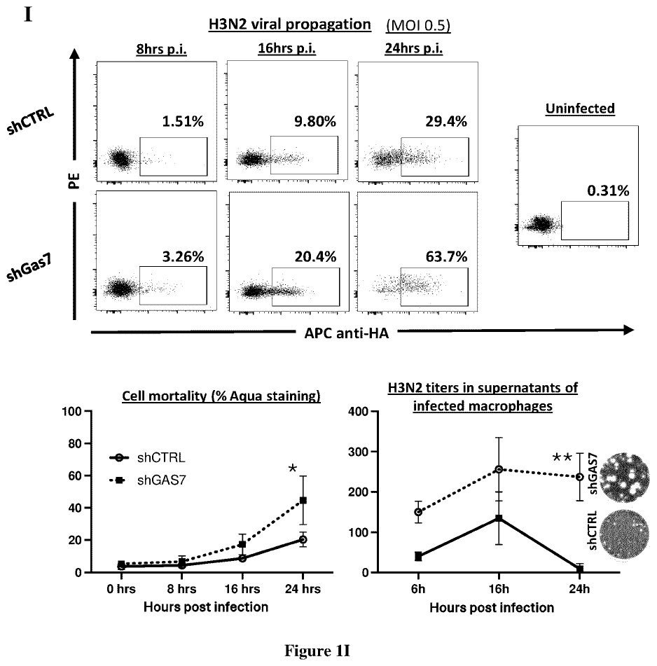

Our work with GAS7 shows that its expression in macrophages limits the replication of viral pathogens belonging to all the major viral groups. We show that the antiviral activity of GAS7 is present even in conditions where the classical antiviral response mediated by type I Interferon is neutralized. This suggests that GAS7 acts via a yet non-characterized mechanism, that we are currently investigating and may involve activation of macrophage autophagy. In particular, we show that in human monocyte-derived macrophages silencing GAS7 boosts the replication of multiple viral pathogens representative of the most relevant viral groups (FIG. 1A-1I). These include the retroviruses HIV-1 and HIV-2 (FIG. 1F), the RNA viruses ZIKA (RNAss+) (FIG. 1H), SINDBIS (RNAss+) (FIG. 1D), Sendai (RNAss−) (FIG. 1G), VSV (RNAss−) (FIG. 1E) and Measles (RNAss−) (FIG. 1C), the influenza viruses (H3N2) (FIG. 1I) and the DNA virus HSV-1 (DNA ds) (FIG. 1B). Importantly, the antiviral activity of GAS7 is present even in conditions where type I Interferon is neutralized, by the addition of the B18R protein (which blocks efficiently type I receptor). Moreover, we show that enforcing the over-expression of GAS7 by macrophages further protects them against HIV-1 infection, when compared with cells expressing normal levels of this factor.

### REFERENCES

Throughout this application, various references describe the state of the art to which this invention pertains. The disclosures of these references are hereby incorporated by reference into the present disclosure.

1. A method of modulating replication of a virus in a population of cells of a subject comprising the step of modulating the expression and/or activity of GAS7 in said population of cells.

2. The method of claim 1 wherein the subject is a human or other animal.

3. The method of claim 1 wherein the population of cells is selected from the group consisting of macrophages, dendritic cells and neurons.

4. The method according to claim 1, wherein the virus is selected from the group consisting of:
coronaviridae;
retroviruses;
flaviviridae;
orthomyxoviruses;
paramyxoviridae;
reoviridae;
picornaviridae;
filoviridae;
arenaviridae;
rhabdoviridae;
herpesviridae;
hepadnaviridae; and
hepeviridae.

5. The method according to claim 1, wherein the virus is a human retrovirus.

6. The method according to claim 1, wherein the step of modulating comprises increasing expression and/or activity of GAS7 in said population of cells.

7. The method of claim 6 wherein the method comprises contacting the population of cells with one or more agent(s) that increase the expression and/or activity of GAS7.

8. The method of claim 7 wherein the agent that increases the expression and/or activity of GAS7 is either i) a polypeptide comprising an amino acid sequence having at least 90% of identity with the amino acid sequence as set forth in SEQ ID NO:1 optionally fused to a cell-penetrating peptide or ii) a polynucleotide encoding for such a polypeptide.

9. The method according to claim 1, wherein the method increases a replication capacity of the virus by contacting the population of cells with an agent that decreases expression and/or activity of GAS7 in said population of cells.

10. The method of claim 9 wherein the agent that decreases the expression and/or activity of GAS7 is either i) a polypeptide comprising an amino acid sequence having at least 90% of identity with the amino acid sequence as set forth in SEQ ID NO:1 optionally fused to a cell-penetrating peptide or ii) a polynucleotide encoding for such a polypeptide is provided that said polypeptide or polynucleotide is a dominant negative polypeptide or polynucleotide of GAS7.

11. The method of claim 9 wherein the agent that decreases the expression and/or activity of GAS7 is a molecule that partially or fully blocks, inhibits, or neutralizes a biological activity or expression of GAS7.

12. The method of claim 11 wherein the agent that decreases the expression and/or activity of GAS7 is selected from the group consisting of antisense polynucleotides, interfering RNAs, catalytic RNAs, RNA-DNA chimeras, GAS7-specific aptamers, anti-GAS7 antibodies, GAS7-binding fragments of anti-GAS7 antibodies, GAS7-binding small molecules, GAS7-binding peptides, and polypeptides that specifically bind GAS7.

13. The method of claim 11 wherein the agent that decreases the expression and/or activity of GAS7 is an endonuclease with designed guide RNA molecules.

14. A method for the prophylaxis or the treatment of a viral infection in a subject in need thereof, comprising administering to the subject an agent that modulates the expression and/or activity of GAS7.

15. (canceled)

16. (canceled)

17. (canceled)


SEQUENCE LISTING (2 sequences)

SEQ ID NO: 1 - protein, 476 aa, Homo sapiens
MSGARCRTLYPFSGERHGQGLRFAAGELITLLQVPDGGWWEGEKEDGLRGWFPASYVQLLEKPGMVPPPPGEESQTVILPPGWQSYLSPQGRRYYVNTTTNETTWERPSSSPGIPASPGSHRSSLPPTVNGYHASGTPAHPPETAHMSVRKSTGDSQNLGSSSPSKKQSKENTITINCVTFPHPDTMPEQQLLKPTEWSYCDYFWADKKDPQGNGTVAGFELLLQKQLKGKQMQKEMSEFIRERIKIEEDYAKNLAKLSQNSLASQEEGSLGEAWAQVKKSLADEAEVHLKFSAKLHSEVEKPLMNFRENFKKDMKKCDHHIADLRKQLASRYASVEKARKALTERQRDLEMKTQQLEIKLSNKTEEDIKKARRKSTQAGDDLMRCVDLYNQAQSKWFEEMVTTTLELERLEVERVEMIRQHLCQYTQLRHETDMFNQSTVEPVDQLLRKVDPAKDRELWVREHKTGNIRPVDMEI

SEQ ID NO: 2 - DNA, 21 bp, synthetic construct
  source 1..21: mol_type = other DNA
gcccagtccaaatggtttgaa